(19) United States
(12) Plant Patent Application Publication
(10) Pub. No.: US 2003/0074705 P1
(43) Pub. Date: Apr. 17, 2003

(54) CREPE MYRTLE PLANT NAMED 'ROSEY CARPET'

(76) Inventors: Robert E. Hambuchen, Conway, AR (US); Betty J. Hambuchen, Conway, AR (US)

Correspondence Address: Christine J. Daugherty, WRIGHT, LINDSEY & JENNINGS LLP, Suite 102, 320 North Rollston Avenue, Fayetteville, AR, 72701, US

(21) Appl. No.: 09/982,461

(22) Filed: Oct. 17, 2001

(51) Int. Cl. A01H 5/00

(52) U.S. Cl. PLT/252

(57) ABSTRACT

A new and distinct variety of the Lagerstroemia genus characterized by a dwarf size, profuse flower production, semi-seedless trait, extensive lateral branching, and lack of continual vertical growth after maturity.

### CROSS-REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE INVENTION

### Field of the Invention

The present invention relates to a new and distinct variety of Lagerstroemia indica ‘prostrata,’ commonly known as crepe myrtle. The novel characteristics of the present invention are its profuse blooms, extended flowering season, semi-seedless trait, heavy branching and ground spreading capabilities, a plant width of 36 to 48 inches, and a low height range of 4 to 12 inches at full maturity, with substantially no vertical growth thereafter. The present invention is more fully described in the following paragraphs and illustrated in the accompanying color photographs.

### BRIEF SUMMARY OF THE INVENTION

Robert E. Hambuchen first discovered the new variety in 1997. The original seed came from Okmulgee, Oklahoma. Thereafter, Robert E. Hambuchen and Betty J. Hambuchen in Conway, Arkansas asexually reproduced the new variety by means of hardwood and softwood cuttings from the original plant.

The new variety is semi-seedless, which causes the plant to express RosePink inflorescences profusely over the entire plant. Unlike other crepe myrtle varieties, which produce inflorescences in a panicle formation near the terminal portion of the stem, this new variety produces rose-pink colored flowers at most leaf nodes and at the terminal portion of most lateral branches. The new variety produces extensive networks of lateral branches, each containing many inflorescences. The flower clusters located on the terminal portion of most lateral branches contain 7 to 20 flower buds and measure approximately 8 to 11 centimeters in height and 5 to 7 centimeters in width. In addition, most leaf nodes contain at least a single flower bud. Because of the extensive lateral branching of this new variety and the presence of the numerous blossoms, an appearance of a rosy pink “foam” of flowers appears over the entire mature plant.

Due to the semi-seedless trait of the new variety, profuse blossoms are noted throughout the growing season. Even in the later portion of the growing season, when most crepe myrtle varieties contain an abundance of seeds and very little blooms, this new variety continues to produce blooms, including blooms at the nodes. The new variety cannot be produced true from seed.

An interesting characteristic of the new variety of crepe myrtle is the very low height range. Fully mature plants reach a maximum height of approximately 12 inches, which produces a heavy branching and spreading crepe myrtle.

The new variety has demonstrated good winter hardiness to approximately 100°F. The new variety also exhibits a high resistance to the powdery mildew fungus.

### BRIEF DESCRIPTION OF THE DRAWINGS

Other distinctive characteristics of the new variety are exemplified in the accompanying photographs. A detailed description of the new variety follows, and, to facilitate identification of the important colors, the terminology adapted by The Royal Horticulture Society Colour Chart has been followed.

FIG. 1 shows the fully mature variety.

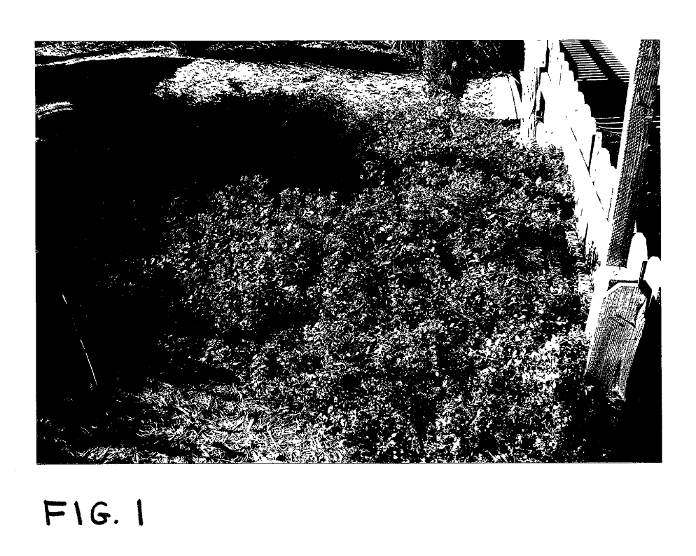

FIG. 2 shows a potted version of the variety grown for five months.

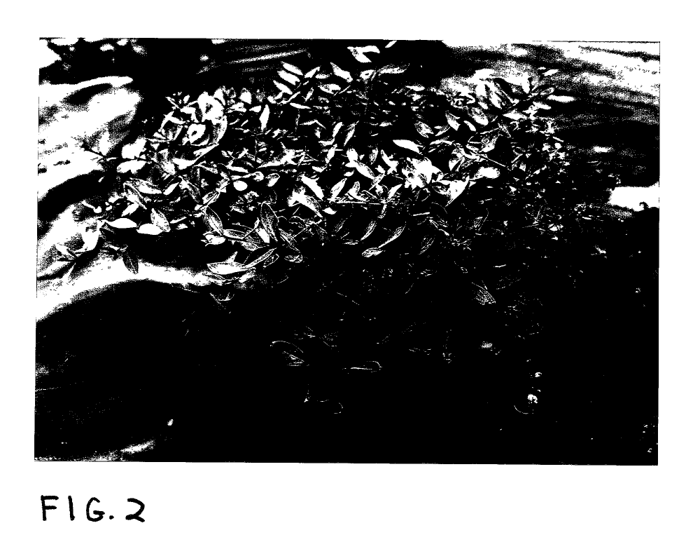

FIG. 3 is a close-up of the fully mature plant with inflorescences.

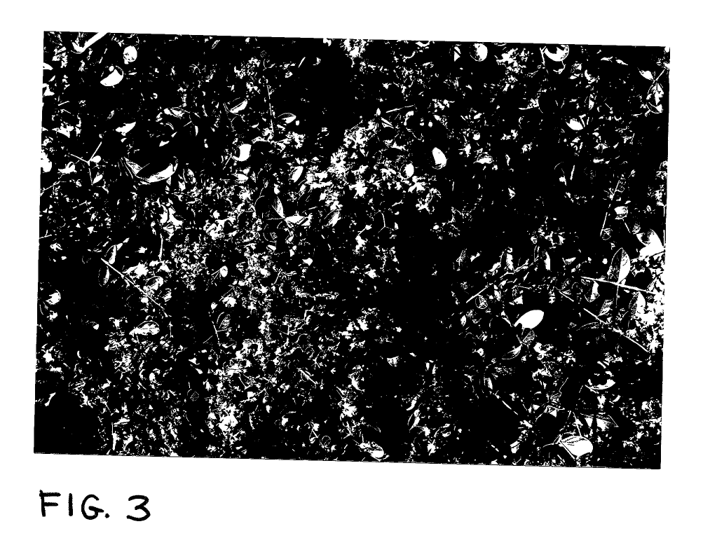

### DETAILED BOTANICAL DESCRIPTION

Parentage: Original seed from Okmulgee, Oklahoma. Original seeds were selected from low growing and spreading plants. Four to five generations of plants exhibiting low growing, weeping and spreading characteristics were screened. The current variety was subsequently selected and asexually propagated by softwood and hardwood cuttings.

Growth: The plant is low to the ground and compact, with a width range of 36 to 48 inches. At maturity, the plant will grow to a height ranging from 4 to 12 inches. There is substantially no vertical growth subsequent to reaching full maturity.

Branches: Multiple branches are freely produced on the young plant. The branches grow horizontally rather than vertically. Branches are a red color when immature and are brown when mature.

Foliage: The leaf arrangement is primarily alternate, with an occasional opposite arrangement. The leaf is an ellipse or ovate shape with both an acute tip and base. The leaves will grow to a length of 2 to 3 centimeters, with a width of 1 to 1.5 centimeters. The leaf attachment is stalked. Leaf color value is reported according to The Royal Horticultural Society Colour Chart code. Mature leaves are colored No. 138 A (light green) on the axial side and colored No. 146 B (yellow green) on the abaxial side.

Flower: The complete flowers are 2 to 3 centimeters high and have a width of 3 to 4 centimeters. Many flowers are arranged in a single bud formation located at the nodes on most stems. The flower clusters are comprised of 7 to 20 flower buds and are located at the terminal portion of most lateral branches. The flower clusters have a width of 5 to 7 centimeters and have a length of 8 to 11 centimeters. The bloom period is from June into October.

Blossom Color: The color of the blossom is Rose Pink No. 61 C. The blossom color value is reported according to The Royal Horticultural Society Colour Chart code.

Seed: The variety is semi-seedless.

We claim:

1. A new distinct variety of Lagerstroemia indica ‘prostrata’ plant substantially as shown and described, characterized by the distinctive Rose-Pink color blooms, profuse flower blossoms, an extended flowering period, semi-seedless trait, extensive lateral branching, a light-green colored leaf, small leaf size, a plant width of 36 to 48 inches, a height range from 4 to 12 inches, and substantially no vertical growth at full maturity.
